(19) United States
(12) Patent Application Publication
(10) Pub. No.: US 2008/0269278 A1
(43) Pub. Date: Oct. 30, 2008

(54) Treatment of Impaired Respiratory Function with Gaboxadol

(75) Inventors: Jonas Lundahl, Hollviken (SE); Bjarke Ebert, Farum (DK); Jan Hedner, Goteborg (SE)

Correspondence Address: DARBY & DARBY P.C., P.O. BOX 770, Church Street Station, New York, NY, 10008-0770, US

(73) Assignee: H. Lundbeck A/S, Valby-Copenhagen (DK)

(21) Appl. No.: 10/599,504

(22) PCT Filed: Mar. 31, 2005
(86) PCT No.: PCT/DK2005/000222

§ 371 Date: Nov. 3, 2006

Related U.S. Application Data
(60) Provisional application No. 60/559,096, filed on Apr. 2, 2004.

(30) Foreign Application Priority Data
Apr. 2, 2004 (DK) 2004 00540

(51) Int. Cl. A61K 31/437; A61P 11/00

(52) U.S. Cl. 514/302

(57) ABSTRACT

The present invention relates to a method for treating impaired respiratory function in a human patient suffering from sleep apnea, such as central sleep apnea or obstructive sleep apnea, comprising administering to said patient an effective amount of gaboxadol per day.

### FIELD OF INVENTION

The present invention relates to the use of gaboxadol for preparing a medicament for treating impaired respiratory function in a human patient suffering from sleep apnea, such as central sleep apnea or obstructive sleep apnea, a method for treating impaired respiratory function in a human patient suffering from sleep apnea.

### BACKGROUND OF THE INVENTION

An open upper airway is a basic requirement for breathing during both wakefulness and sleep. Intensive research during the last two decades has led to the identification of a complex of conditions characterized by inadequately low airway patency—and thus breathing—which occur exclusively during sleep. In patients suffering from one or more out of several forms of these condition, breathing becomes partially or totally interrupted during sleep due to a collapse or obstruction of the upper airway.

The presently applied principal forms of treatment in sleep apnea include surgery of the upper airway, intraoral mandibular advancement devices and long-term treatment with nasal continuous positive airway pressure (nCPAP). These methods of treatment are cumbersome, poorly tolerated and/or expensive. Various forms of pharmacological treatment, e.g. by administration of tricyclic antidepressants, selective serotonin reuptake inhibitors and progesterone have been employed but have not gained wide clinical use due to limited efficacy. The respiratory stimulant theophylline and azetazolamide, an carbonic anhydrase inhibitor, have experimentally been employed in various forms of central sleep apnea (CSA) but are not applied in the clinical routine.

In WO 00/51590 various mechanistic approaches to preventing or ameliorating sleep-related breathing disorders are presented. It is also alleged that amongst others GABA receptor agonists could be useful, and as examples are mentioned isoguvacine, muscimol, THIP, piperidine-4-sulphonic acid, flunitrazeparn, zolpidem, abecarnil, baclofen, piracetam, and progabide. However, several of these are not even GABA receptor agonists. Gaboxadol (4,5,6,7-tetrahydroisoxazolo(5,4-c)pyridin-3-ol) described in EP patent 0000338 B1, and in EP Patent 0840601 B1 has shown great potential in the treatment of sleep disorders in general.

### DESCRIPTION OF THE INVENTION

There is a need for a new effective treatment of impaired respiratory function in a human patient suffering from sleep apnea. In particular, pharmacological treatment of such disorders would offer a definite advantage over the invasive or non-invasive methods used at present, many of which only provide insufficient relief and some of which are cumbersome to the patient.

Human patients suffering from sleep apnea may suffer from depression at the same time, or depressed people may actually develop sleep apnea. Thus, we also believe there is a special need for treating this group of patients that suffer from sleep apnea and depression at the same time.

Apnea specialists generally agree that there are three different types of sleep apnea: obstructive, central, and mixed. Of these three, obstructive sleep apnea (OSA) is the most common; central sleep apnea (CSA) is rare; mixed sleep apnea is a combination of the previous two with treatment being the same as OSA.

Obstructive sleep apnea is characterized by repetitive pauses in respiration during sleep due to the obstruction and/or collapse of the upper airway (throat), usually accompanied by a reduction in blood oxygen saturation, and followed by an awakening to breathe. This is called an apnea event. Respiratory effort continues during the episodes of apnea. An analogy might be helpful: OSA is like putting your hand over your vacuum cleaner intake nozzle. Your hand blocks all air from getting through (upper airway collapse) even though the vacuum cleaner is still applying suction (respiratory effort continues). The vacuum cleaner is usually straining somewhat at this time, and so does the human body.

Central Sleep Apnea is defined as a neurological condition causing cessation of all respiratory effort during sleep, usually with decreases in blood oxygen saturation. To return to the vacuum cleaner analogy: central sleep apnea would be like pulling the plug on the vacuum cleaner. No power, no suction: if the brainstem center controlling breathing shuts down there's no respiratory effort and no breathing. The person is aroused from sleep by an automatic breathing reflex, so may end up getting very little sleep at all.

It should be observed that obstruction, in the context of the present invention, excludes obstruction by foreign objects or by material excreted by the body, such as mucus. In its simplest form partial airway collapse or obstruction is indicated by profound and vigorous snoring. More prominent airway collapse or obstruction results in so called hypopnea, a condition in which airflow is significantly reduced during inspiration with or without concomitant signs of hypoxemia. The most severe form, obstructive apnea, describes a state of total collapse of the upper airway. The condition, in its more pronounced forms, is associated with repeated episodes of interrupted airflow during which the patient maintains inspiratory attempts against an occluded airway. The reduction of airflow eventually leads to hypoxemia, hemodynamic changes and arousal from sleep. Moreover, cardiovascular complications are common in obstructive sleep apnea. Obstructive sleep apnea has been associated with increased insulin resistance, diabetes, obesity, alterations of lipid metabolism and increased platelet aggregability. It is important to point out that the symptoms and complications listed above are not confined to severe cases. They may also be observed in cases of partial sleep apnea characterized by frequent hypopneas or even intense snoring.

A number of factors that predispose for airway collapse during sleep have been identified. Among others these include obesity, hypertrophied upper airway tissue (particularly in children), and short jaw. However, a substantial number of subjects with mild, moderate or severe sleep apnea do not exhibit any of these factors and may therefore be referred to as cases with essential sleep apnea. It appears likely that essential sleep apnea may be caused by central nervous mechanisms relating to reduced nervous activity to upper airway muscles responsible for maintenance of upper airway aperture during sleep. Such mechanisms may also be important for precipitating or aggravating sleep disordered breathing in cases with predisposing factors such as described above.

The absence of obvious aberrant anatomic factors, however, does not exclude a dynamic malfunction of the tongue and the upper airway dilating musculature. Such defect function may originate in the central nervous system, at the level of signal transmission to peripheral muscles or at the neuromuscular junction. Such defective control seems to be particularly pronounced during sleep only, suggesting the central nervous, peripheral neural and/or neuromuscular control of the upper airway is particularly prone to be affected in this state. Moreover, and importantly, upper airway tone will ultimately counteract the airway collapsing forces generated by inspiratory flow of air in the airway. A situation characterized by pronounced chemoreflex activation and thereby high ventilatory drive in order to optimize the opportunities for a high inspiratory flow. If this potentially collapsing force is counteracted by inappropriately low airway tone the airway will tend to collapse. This potential mechanism is especially attractive in a specific form of sleep disordered breathing referred to as central apnea, periodic breathing and/or Cheyne-Stokes respiration (all here referred to as central sleep apnea). This form of breathing disorder is characterized by an oscillating pattern of respiration which periodically is driven by considerable chemoreflex activation.

Ventilatory control stability depends on several factors involved in the loop of events responsible for maintenance of metabolic homeostasis. This loop includes a central controller gain (including chemoreceptor responsiveness, brain stem respiratory center responsiveness and excitability) and a series of plant factors that determine the extent to which gas tensions in mixed pulmonary capillary blood will change for a given change in ventilation. Finally there are factors which will determine the change in gas tension at the chemoreceptor for a given change in pulmonary capillary gas tension. These factors include pulmonary circulatory delays and diffusion delays which are involved in the process of the chemoreflex feedback. The “loop gain” or extent of feedback control which includes all these factors provides a substrate for estimation of the susceptibility to periodic breathing. When loop gain is changed to a value of unity instability can occur and this change may take place at any of the steps included in the loop. In this sense it may be advocated that different forms of sleep apnea including central and obstructive forms may have a similar principal pathogenetic background. In some cases gain may be altered substantially without development of sleep disordered breathing suggesting a highly stable respiratory control system. In others central apneas may be elicited after minor alteration of gain suggesting a control system prone to oscillation. Such increased proneness may be more prevalent in for instance cardiac failure and in patients with compromised upper airway aperture highly dependent on upper airway dilatory muscle activity for maintenance of ventilation. Moreover, sleep per se, particularly REM sleep, appears to be a particularly prominent modifier of loop gain in certain patients with sleep disordered respiration. From this reasoning it follows that a remedy that modifies one or several of the elements encompassed in the loop system may effectively alter the characteristics of the loop and reduce or eliminate sleep disordered respiration.

An objective of the invention is to provide an effective treatment of impaired respiratory function in a human patient suffering from sleep apnea, in particular central sleep apnea or obstructive sleep apnea or a mix thereof, which reduces and/or eliminates some or all of the drawbacks of the methods unknown to the art.

A further objective of the invention is to provide an effective treatment, in particular long-term treatment, of a human patient suffering from sleep apnea.

A further objective of the invention is to provide an effective treatment, in particular long-term treatment, of a human patient, without causing abuse or dependency of treatment.

A further objective of the invention is to provide an effective treatment of human patients that suffer from sleep apnea and depression at the same time.

Further objectives of the invention will become apparent upon reading the present specification.

Gaboxadol has the general formula

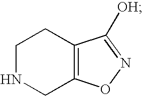

and throughout the description “gaboxadol” is intended to include any form of the compound, such as the base (zwitter ion), pharmaceutically acceptable salts, e.g. pharmaceutically acceptable acid addition salts, hydrates or solvates of the base or salt, as well as anhydrates, and also amorphous, or crystalline forms.

Treatment of impaired respiratory function is intended to mean improving or alleviating, the respiratory function in patients suffering from sleep apnea, over a period of sleep, such as 10 minutes to 10 hours.

The treatment is typically given during less than a week (short term treatment), from 1 to 4 weeks (intermediate term treatment) or for a period exceeding 4 weeks (long-term treatment). A special type of long-term treatment is chronic treatment.

The term “elderly” is intended to mean humans from 65 years and above.

The term “adults” is intended to mean humans from 18 to 64 years.

According to the present invention an effective medicament with no significant side-effects for the treatment of impaired respiratory function in a human patient suffering from sleep apnea, such as central sleep apnea or obstructive sleep apnea is provided.

In a broad aspect, the present invention relates to use of gaboxadol for preparing a medicament for treating sleep apnea in a human patient.

In another aspect, the present invention relates to use of gaboxadol for preparing a medicament for treating impaired respiratory function in a human patient suffering from sleep apnea, such as central sleep apnea or obstructive sleep apnea.

In one embodiment, sleep apnea is a mixture of central sleep apnea and obstructive sleep apnea.

In a further embodiment the human patient suffers from depression and sleep apnea at the same time.

In another embodiment, gaboxadol increases slow wave sleep in the patient and thereby improves the respiratory function.

In a further embodiment, gaboxadol is in the form of an acid addition salt, or a zwitter ion hydrate or zwitter ion anhydrate. In a further embodiment, gaboxadol is in the form of the pharmaceutically acceptable acid addition salt selected from the hydrochloride or hydrobromide salt, or in the form of the zwitter ion monohydrate.

In a further embodiment, the medicament is an oral dose form. Typically, the medicament is a solid oral dose form, such as tablets or capsules, or a liquid oral dose form. Thus, a typical embodiment is use of gaboxadol for preparing a medicament in an oral dose form comprising an effective amount of the gaboxadol from 2.5 mg to 20 mg, for treating impaired respiratory function in a human patient, such as an elderly human patient. The effective amount ranges from 2.5 mg to 20 mg of gaboxadol calculated as the base. Preferably, the gaboxadol is in a crystalline form. Further embodiments of the medicament comprises an effective amount of gaboxadol from 2.5 mg to 20 mg, such as 2.5 mg to 4 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg, e.g. 2.5 mg, 5 mg, 7.5 mg, 10 mg, 12.5 mg, or 15 mg. A typical embodiment being 5 mg to 15 mg of crystalline gaboxadol, such as the hydrochloride of gaboxadol.

The human patient to be treated with gaboxadol may in fact be any subject of the human population, male or female, which may be divided into children, adults, or elderly. Any one of these patient groups relates to an embodiment. Typically, the human patient is selected from adults or elderly patients.

In a further embodiment, the treatment is short term treatment. In a further embodiment the treatment is intermediate term treatment. In a further embodiment the treatment is long term treatment. In a further embodiment the treatment is chronic treatment.

A typical embodiment is use of gaboxadol for preparing a medicament, such as in an oral dose form, comprising an effective amount of the gaboxadol from 2.5 mg to 20 mg, for long term treatment of impaired respiratory function in a human patient, such as an elderly human patient, suffering from sleep apnea, such as central sleep apnea or obstructive sleep apnea.

In a farther aspect, the present invention relates to a method for treating sleep apnea in a human patient, comprising administering to said patient an effective amount of gaboxadol per day. In a still further aspect the present invention relates to a method for treating impaired respiratory function in a human patient suffering from sleep apnea, such as central sleep apnea or obstructive sleep apnea, comprising administering to said patient an effective amount of gaboxadol per day. Typically, the effective amount in an oral dose form, comprises gaboxadol from 2.5 mg to 20 mg per day.

The timing of the administration of gaboxadol according to the invention will depend on the formulation and/or route of administration used. Typically, administration of gaboxadol will, in the majority of cases, be given as a long-term treatment regimen whereby pharmacokinetic steady state conditions will be reached. Medication for peroral or parenteral administration may also be given in immediate relation to a particular sleeping period, for instance 10 minutes to 3 hours prior to the onset of sleep. Thus, when using gaboxadol for preparing a medicament, or when administering gaboxadol, a typical embodiment is an oral medicament, or peroral administration, wherein gaboxadol is given in immediate relation to a particular sleeping period from 5 minutes to 5 hours prior to onset of sleep, such as 10 minutes to 3 hours prior to the onset of sleep.

In a further aspect, the present invention relates to use of gaboxadol for preparing a medicament comprising an amount of from 2.5 mg to 20 mg of gaboxadol for treating sleep apnea in a human patient, said amount being effective during a substantial portion of a single sleep period.

In a still further aspect, the present invention relates to use of gaboxadol for preparing a medicament comprising an amount of from 2.5 mg to 20 mg of gaboxadol for treating impaired respiratory function in a human patient suffering from sleep apnea, such as central sleep apnea or obstructive sleep apnea, said amount being effective during a substantial portion of a single sleep period.

In a further aspect, the present invention relates to a method for treating impaired respiratory function in a human patient suffering from sleep apnea, such as central sleep apnea or obstructive sleep apnea, comprising administering to said patient an effective amount of 2.5 mg to 20 mg gaboxadol per day, said amount being effective during a substantial portion of a single sleep period.

In a further embodiment, the substantial portion is 40% or more, 50% or more, 60% or more, 70% or more, such as 80% or more.

In a further embodiment, the single sleep period is from one to eight hours. Typically, the single sleep period is from one to four hours, or from one to six hours, such as 1, 2, 3, 4, 5, 6, 7, or 8 hours.

In a further embodiment, the amount of gaboxadol is released from a composition for controlled release, such as an extended release composition.

In a further embodiment, from 50% to 100% of the amount of gaboxadol is released within a period of three hours from administration.

In a further embodiment, from 80% to 100% of the amount of gaboxadol is released within a period of five hours from administration.

According to the invention gaboxadol may be used as the base (i.e. the zwitter ion) or as a pharmaceutically acceptable acid addition salt thereof or as an anhydrate or hydrate or solvate of such salt or base. The salts of the compound used in the invention are salts formed with ion-toxic organic or inorganic acids. Exemplary of such organic salts are those with maleic, fumaric, benzoic, ascorbic, succinic, oxalic, bis-methylenesalicylic, methanesulfonic, ethane-disulfonic, acetic, propionic, tartaric, salicylic, citric, gluconic, lactic, malic, mandelic, cinnamic, citraconic, aspartic, stearic, palmitic, itaconic, glycolic, p-amino-benzoic, glutamic, benzene sulfonic and theophylline acetic acids, as well as the 8-halotheophyllines, for example 8-bromo-theophylline. Exemplary of such inorganic salts are those with hydrochloric, hydrobromic, sulfuric, sulfamic, phosphoric and nitric acids. Gaboxadol may also be used as the zwitter ion, e.g. the monohydrate thereof.

The acid addition salts according to the invention may be obtained by treatment of gaboxadol with the acid in an inert solvent followed by precipitation, isolation and optionally re-crystallisation by known methods and if desired micronization of the crystalline product by wet or dry milling or another convenient process, or preparation of particles from a solvent-emulsification process. Suitable methods are described in EP patent 0000338.

Precipitation of the salt is typically carried out in an inert solvent, e.g. an inert polar solvent such as an alcohol (e.g. ethanol, 2-propanol and n-propanol), but water or mixtures of water and inert solvent may also be used.

According to the invention, gaboxadol should be administered orally, and it may be presented in any suitable form for such administration, e.g. in the form of tablets, capsules, powders, syrups or solutions. Typically, and in accordance with the purpose of the present invention, gaboxadol is administered in the form of a solid pharmaceutical entity, suitably as a tablet or a capsule.

Methods for the preparation of solid pharmaceutical preparations are well known in the art. Tablets may thus be prepared by mixing the active ingredients with ordinary adjuvants and/or diluents and subsequently compressing the mixture in a convenient tabletting machine. Examples of adjuvants or diluents comprise: corn starch, lactose, talcum, magnesium stearate, gelatine, lactose, gums, and the like. Any other adjuvant or additive such as colourings, aroma, preservatives, etc. may also be used provided that they are compatible with the active ingredients.

A suitable formulation of gaboxadol is described in WO 02/094225 filed May 17, 2002. Without limiting the invention in any way, it is intended that any one of the aspects or embodiments of this patent application is suitable embodiments of the medicament or pharmaceutical compositions herein.

### EXPERIMENTAL PROCEDURE

Human patients suffering from sleep apnea are dosed prior to bedtime with gaboxadol p.o. at doses from 2.5 mg to 20 mg.

1-22. (canceled)

23. A method for treating impaired respiratory function in a human patient suffering from sleep apnea comprising administering to said patient an effective amount of gaboxadol per day.

24. A method for treating sleep apnea in a human patient, comprising administering to said patient an effective amount of gaboxadol per day.

25. The method of claim 23, wherein the sleep apnea is central sleep apnea.

26. The method of claim 23, wherein the sleep apnea is obstructive sleep apnea.

27. The method of claim 23, wherein the sleep apnea is a mix of central sleep apnea and obstructive sleep apnea.

28. The method of claim 23, wherein the gaboxadol increases slow wave sleep in the patient and thereby improves the respiratory function.

29. The method of claim 23, wherein the human patient suffers from sleep apnea and depression at the same time.

30. The method of claim 23, wherein the gaboxadol is in the form of an acid addition salt, a zwitter ion hydrate, or a zwitter ion anhydrate.

31. The method of claim 23, wherein the gaboxadol is in the form of its hydrochloride or hydrobromide salt.

32. The method of claim 23, wherein the gaboxadol is in the form of its zwitter ion monohydrate.

33. The method of claim 23, wherein the gaboxadol is administered orally.

34. The method of claim 23, wherein the gaboxadol is administered in the form of an oral dosage form.

35. The method of claim 34, wherein the oral dosage form is a solid dosage form.

36. The method of claim 35, wherein the oral dosage form is a tablet or capsule.

37. The method of claim 34, wherein the oral dosage form is a liquid dosage form.

38. The method of claim 34, wherein the oral dosage form comprises from 2.5 mg to 20 mg of gaboxadol.

39. The method of claim 23, wherein the human patient is selected from elderly or adults.

40. The method of claim 23, wherein said treatment is intermediate term treatment.

41. The method of claim 23, wherein said treatment is short term treatment.

42. The method of claim 23, wherein said treatment is long term treatment.

43. The method of claim 23, wherein said gaboxadol is crystalline.

44. The method of claim 34, wherein the dosage form comprises an amount of from 2.5 mg to 20 mg of gaboxadol, said amount being effective during a substantial portion of a single sleep period.

45. The method of claim 44, wherein the dosage form comprises 5 mg to 15 mg of gaboxadol.

46. The method of claim 44, wherein said substantial portion is 50% or more.

47. The method of claim 46, wherein said substantial portion is 80% or more.

48. The method of claim 44, wherein said single sleep period is from one to eight hours.

49. The method of claim 44, wherein the amount of gaboxadol is released from a composition for controlled release.

50. The method of claim 49, wherein from 50% to 100% of the amount of gaboxadol is released within a period of three hours from administration.

51. The method of claim 49, wherein from 80% to 100% of the amount of gaboxadol is released within a period of five hours from administration.
